(19) United States
(12) Patent Application Publication
(10) Pub. No.: US 2013/0324919 A1
(43) Pub. Date: Dec. 5, 2013

(54) Strapping Device for Use with Breast Pumps

(71) Applicant: Kami L. Gallaher, San Francisco, CA (US)

(72) Inventor: Kami L. Gallaher, San Francisco, CA (US)

(21) Appl. No.: 13/484,551

(22) Filed: May 31, 2012

(51) Int. Cl. A61M 1/06

(52) U.S. Cl. 604/74

(57) ABSTRACT

A single piece strapping device that can be used with breast pumps. The strapping device has two ends, with each end having an attachment; the two attachments can be connected together such that the strapping device becomes a single piece unit surrounding the torso of the user. The strapping device has two openings, provided thus to attach funnel shaped components of the breast pumps onto each breast.

### FIELD OF THE INVENTION

The present invention generally relates to breast milk pumps, and more particularly relates to attaching the devices to the breasts during usage. More specifically, the invention relates to an attachment that comprises a strap for securing the funnels directly to the breast, freeing up the hands of the user from having to hold the device while the breast milk pump is in operation.

### BACKGROUND OF THE INVENTION

Lactating mothers who cannot feed their babies directly use Breast milk pumps as a means to collect and store the breast milk for usage later. Breast milk pumps are well known and widely used; and generally comprise of a vacuum pump, a funnel type attachment to each breast, receptacles for collecting and storing the milk pumped and connecting tubes for carrying the milk from the funnels to the receptacles. The vacuum pump itself may be hand driven or driven by a motor to create intermittent suction, so that the operation simulates the sucking action created by the baby during direct breast feeding. The funnel is designed in such a way as to cover the breast as a whole, with the central circular portion of the funnel not touching the breast. This region leaves the nipple and part of the immediately surrounding areola region untouched during the pumping operation. The remaining part of the funnel, which touches the breast, forms a circular ring region, which presses the areola region and the surrounding tissues of the breast during the vacuum application to release milk through the nipple. It is important that the funnel shaped attachment seals this region, as the Montgomery's glands in this region provide lubrication necessary to protect the breast surface from physical damage during the pumping operation. This region would then simulate the lips and gum region of the baby, so that a seal is provided for the pump to effectively suck milk through the nipple openings.

The pumping action being intermittent application of vacuum, it is imperative that the holding device, which is typically funnel shaped, be secured to the breast well so that the seal is not broken during the on and off cycling of the vacuum. Typically, the person holds these devices with both hands to ensure that the vacuum seal is not broken during the cyclical pumping operation. This is not only physically challenging, but also difficult to continuously achieve. The hands can get tired and may also move or shake, removing the vacuum and also damaging the skin surface. To free up the persons hands and in order to ensure continuous and effective seal for the vacuum, it is highly beneficial to have a device that holds the funnel shaped devices to the breasts continuously and completely. It is also possible that when the vacuum is lost, the collected milk could spill and be lost. Having a device that provides continuous and complete support can prevent this from happening, saving milk that is precious for the baby.

One of the common approach is to use a string that goes around the neck and holds the funnel in place. This string is fairly easy to use, and also to adjust the length of, and ensures that the funnels do not tip forward and fall. In this approach, a loop is available to pass over each funnel, which can be adjusted to fit the size of the funnel end. This approach has the limitation that it does not provide the pressure needed to ensure that the vacuum seal remains throughout the pumping session, especially when the vacuum is low or non-existent during the pumping cycle. Another limitation of the approach is that the point of contact between the string and the funnel is minimal and is mainly intended to provide holding support in the vertical direction. Yet another limitation of this approach is that as explained in the previous paragraph, the approach does not provide continuous and complete support, and actions such as sneezing, coughing or even leaning forward can limit the efficiency of the string in providing support.

Another approach is the use of a multi piece strap that goes around the body, under both the arms. This approach uses a wider device, like a band, that is used when compared to the string used in the previous approach. In this approach, two sets of hooks or a hook and loop type fastener (more commonly known as velcro), one in the back side and one between the breasts is used to locate and hold the funnels in place. In the second approach, cuts or openings are provided in each piece of the strap for each funnel to fit in. This approach provides the pressure needed on the funnels to hold them in place compared to the strings, and work fairly well in the low or no vacuum stages of the pumping cycle. However, the openings are typically just cuts in the strap cloth. These cuts in the cloth or textile material often create loose fibers. The loose fibers, or fiber ends, can end up between the funnel and the breast or between the funnel and the connecting tube, which can break the vacuum seal. Further, small pieces of these fibers can even end up in the milk collected, which can potentially choke or become hazardous when the milk is later fed to the infant. The cut in the fabric can also be an initiating spot for further tear of the strap material, thus changing the size and location of the openings after being used multiple times.

It is also well known that the breast changes size and contours during the months of lactation as the baby's feeding habits change and reduce over time. The breasts can become engorged during times and days of limited feedings. From the beginning of one pumping session to its end, the breast reduces in size as milk is removed from it. Eventually, as the baby reduces taking breast milk and switches to solid foods, the breasts slowly return to a smaller size. To accommodate these changes in contours and sizes, and also to accommodate users with various breast sizes, breast milk pump manufacturers provide funnels with various sizes. Hence, it is preferred that a strapping device be universal if possible. A simple and efficient design for the strap is thus advantageous in that the same product can be used under all circumstances; and even by multiple users over the lifetime of the strap itself. A single design that can meet these varying needs is also more economical to produce by manufacturers as well as stock by distributors and sellers, effectively making it cheaper for the end user to procure and use.

The size and shape of the areola has been mentioned before as important. The funnel shaped attachment to the breast needs to cover this region in contact area. More importantly, a strap that is used to hold the funnels in place need to ensure that pressure is applied at the right regions in the funnel. The application of the pressure at the right locations is important not only to ensure that the vacuum is sustained during pumping, but also to ensure that the milk from the milk ducts in the breasts are squeezed out during the pumping operation. The size, location and shape of the openings in the strap are hence of utmost importance to ensure optimal support and pressure.

Applicant believes that her invention provides a device that meets all the above mentioned needs of a strapping device.

### SUMMARY OF THE INVENTION

The current invention is a single piece strapping device that can be used to locate the funnel shaped attachments, which are typically used with breast pumps, onto the breasts, and to hold them in place during the pumping operation, while providing adequate pressure to the surrounding areas of the nipple to ensure proper vacuum seal and pressure on the milk ducts, while also being universal in design to be used on a wide range of breast sizes and shapes.

One of the principal objects of the present invention is to provide a single piece device which can be used for the attachment of the funnels of the breast pump onto each of the breasts.

Another aspect of the present invention is the incorporation of two openings in the device. The openings can be spaced anywhere from 5 centimeters to 50 centimeters apart, when measured from center to center, and more preferably be spaced between 10 and 40 centimeters apart. The openings themselves can be anywhere from 2 centimeters to 20 centimeters in size, when measured from one end to the other, and more preferably be between 5 and 15 centimeters in size. It has been found that when the openings are sized and spaced within the above limits, optimum holding pressure is obtained on the funnels, thus ensuring both excellent retention of the vacuum seal and optimal pressure on the breast itself to ensure optimal pumping effect.

Yet another aspect of the present invention is the use of piping around the opening edges described earlier. Piping is a well known approach used in clothing, textiles and upholstery to secure ends of the material. In the current invention, use of piping has been found to be extremely effective in eliminating loose fiber ends which can prevent the vacuum seal between the funnel and the breast or between the funnel and the connecting tubing. The use of piping, especially at slightly larger sizes than the strap material itself, has been found to be effective in distributing the pressure from the strap all around the nipple in an evenly manner. The piping effectively acts as the main region of contact between the strap and the funnel, thus distributing pressure more evenly on the areola region of the breast. This also increases the comfort of the user.

The strap further incorporates an attaching mechanism to hold itself around the body of the person using it. This attachment design ensures that the openings are located exactly on top of each breast and that the funnel shaped devices can be attached onto each breast through the two openings provided in the strap. The preferred mode of attachment is the use of a Velcro, while it is also possible to use the device with a hook, button or other forms of attachments. The preferred location of the attachment in the strap is on the backside of the person, while it is also possible to alter the design to have the attachment made in the front between the two breasts or even under one of the arms or on the side of the body.

FIG. 1 and FIG. 2 provide the design of such a strap. The strap, 10, as shown, has two openings, 26 and 33. Male and female components of the attaching device, 48 and 49 are placed on either end of the strap, one on the inside and one on the outside. The strap is used by placing it on top of the breasts and wrapping the ends around the torso with the two attaching device components being connected together where the ends of the strap will overlap each other. FIG. 3 provides a close up view of one of the openings, the piping around the opening, 57 being shown. The piping is characteristically thicker than the base strap material. Optionally, piping can also be provided all around the strapping device to improve aesthetic appeal.

The accompanying drawings and the following detailed description of an embodiment of the invention will together allow further appreciation of the present invention, its attributes and advantages.

### BRIEF DESCRIPTION OF THE DRAWINGS

FIG. 1 is a front view of an embodiment of a strapping device used for attaching parts of the breast pump onto the breasts

FIG. 2 is a back view of an embodiment of a strapping device used for attaching parts of the breast pump onto the breasts

FIG. 3 is a close up view of one of the openings marked in FIG 1 and FIG 2.

### DETAILED DESCRIPTION OF EMBODIMENTS OF THE INVENTION

The embodiments of the invention described hereinafter have been particularly adapted for use with a conventional, commercially available breastpump; it need not be commercial or conventional, as long as the pump has funnel shaped attachments that needs to be attached to the breasts of the user prior to the usage of the breast pump itself. The breastpump may be manual or motor driven.

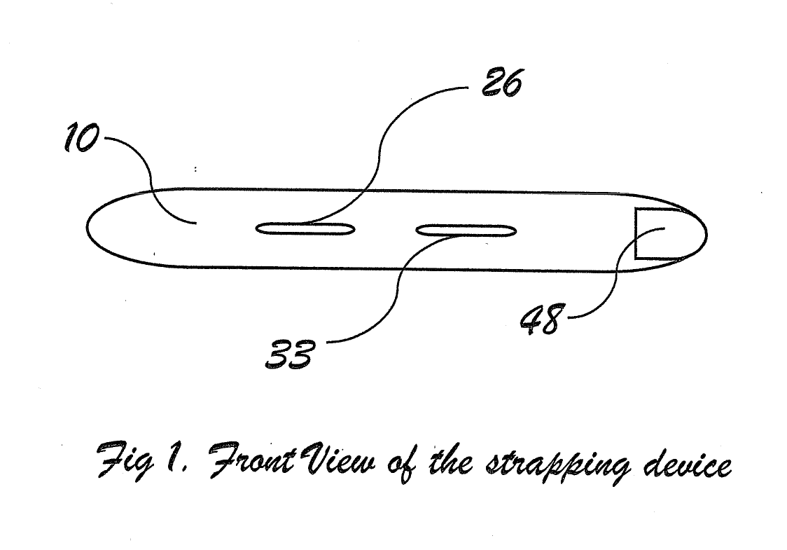

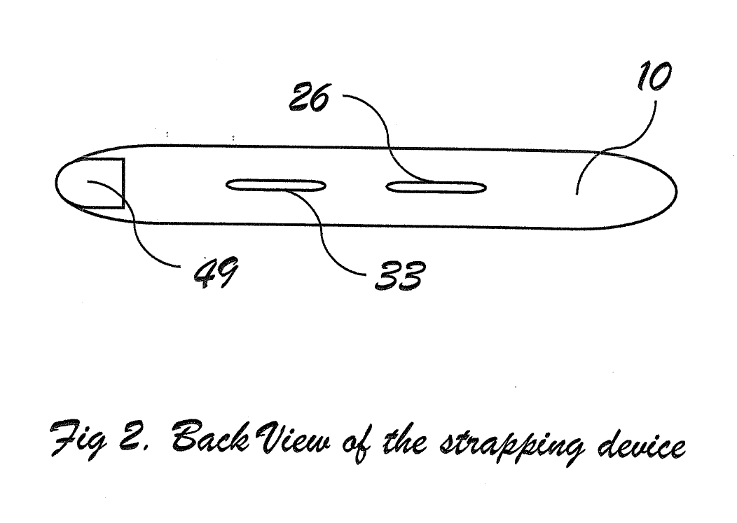

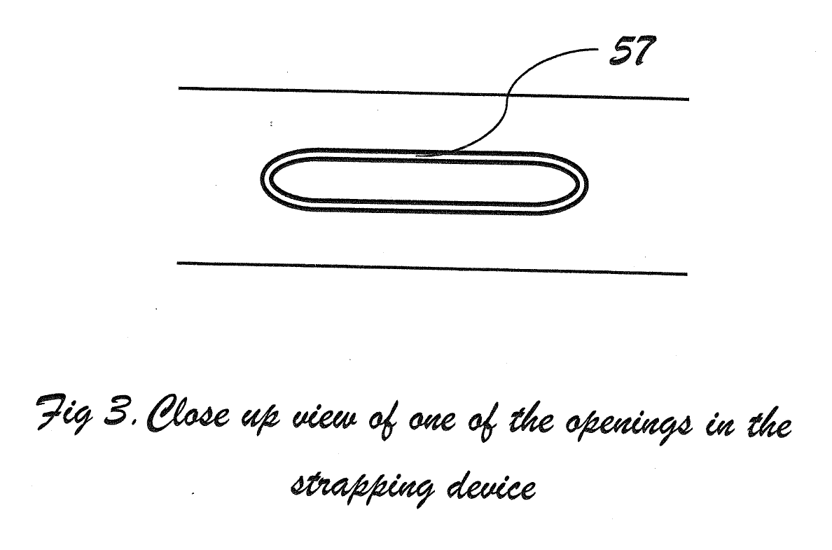

Referring to FIG. 1 and FIG 2, the front and back views of the strapping device are shown. The device comprises a long single piece unit, 10, with two openings, 26 and 33. FIG. 1 shows one of the attachments, 48, on one of the ends of the strapping device, on the front side. FIG 2 shows the back side of the strapping device, and shows the second part of the attachment, 49, at the other end of the strapping device. Piping that goes around the opening, 57, is shown in FIG 3, a close up view of one of the openings.

In one embodiment, the strapping device is a long single piece unit, and can be made using suitable materials well known to those in the art, including, but not limited to, different textile materials such as cotton, wool, silk, nylon, polyester, lycra®, other such fibrous materials, or mixtures thereof. One of the preferred materials for making the strapping device is spandex®. Another preferred material is a mix of nylon, lycra®, cotton and neoprene.

In one embodiment, the strapping device has two ends, and each of the ends have attachments that can be connected to each other. The attaching device can be of various forms, and these are well known to those in the art. Typical attaching devices include hooks, velcro, buttons, press buttons, zips and clips. Other simpler or more complex attaching devices can also be used as desired. Of the mentioned attaching devices, velcro is one of the preferred options to use. In using a velcro, the hook and loop parts of the velcro are attached to either ends of the strapping device, one on the front side of the device and one on the back side of the device. This design makes it possible for the strapping device to be placed over the breasts of the user with the two ends going under each of the arms and overlapping on the backside of the torso of the person, in such a manner that the hook and loop parts of the velcro will connect to each other and provide a secure strapping action. Another preferred attachment mechanism is hooks.

In another embodiment, the strapping device has two openings, provided therein to facilitate attaching parts of the breast pump to each of the breasts. The two openings shown in FIG. 1 and FIG. 2 are designed in size and shape to support the funnel shaped attachments. Breast pumps that are available in the market have funnel shaped attachments that are placed over each breast, and these are further connected to bottles or similar collecting containers using tubes or similar connecting mechanism. The breast pump manufacturer provides the funnel shaped attachment in different sizes to accommodate needs of users with different breast sizes. The strapping device described in this invention provides a comfortable, easy to use and simple approach to secure these funnel shaped devices onto the breasts, thus freeing up the hands of the user. The strapping device has two openings to accommodate the funnel shaped devices. The openings can be shaped as circles, ovals, rectangles, squares, diamonds, hearts, stars, dumb-bells, flowers, or other shapes. Preferably, the openings are long in shape, the longer dimension of the opening matching the longer dimension of the strapping device. The sharp corners can act as initiators for tears. It is hence preferable that the openings have only curved ends, so shapes with sharp corners such as squares, rectangles or diamonds are less preferred. In case such shapes are used, the corners can be rounded to eliminate the potential for tears initiating from these locations. Long ovals are one of the preferred shapes for the openings. Another preferred shape for the openings is a long thin rectangle.

In yet another embodiment of the present invention, the openings are sized between 5 and 50 centimeters apart, when measured from center of first opening to the center of the second opening. The two openings have to be spaced such that the funnels can be placed exactly at the center of each of the breasts, yet the funnel ends can pass through the openings in the strapping device for easy attachment to the connecting devices that carry the breast milk from the funnel ends to the collecting containers such as bottles. It is further preferred that the openings are spaced between 10 and 40 centimeters apart.

In yet another embodiment of the present invention, each of the two openings in the strapping device are between 2 and 20 centimeters in length. An opening of this size permits the funnel shaped attachment of the breast pump to go through the opening easily. The size of the opening is also important in ensuring that during the usage, the pressure from the strapping mechanism is uniformly and evenly distributed around the nipple area of the breast, and on the region of the breast where the milk ducts are present. The size and shape of the opening have to match those of the areola region of the breast. In such a situation, the pressure is applied uniformly around the nipple, and optimal release of milk during the pumping action of the breast pump by use of intermittent vacuum in made possible. It is further preferred that each of the openings are between 5 and 15 centimeters in length.

In yet another embodiment of the present invention, each of the openings is designed to be in parallel direction of the strapping device. By this, it is meant that the lengthwise direction of the strapping device and the openings match each other. The width direction of the strapping device and the openings further match each other. When the strapping device is in use, with the attachments at either end being connected to each other with the strapping device going around the torso of the user, the strapping forces are in the lengthwise direction of the strapping device. The strapping device and the forces are in the horizontal direction, when the user is standing or sitting erect. Designing the openings in a vertical direction or perpendicular to the length of the strapping device will create high stress points at either ends of the opening, which can then initiate tears. When the opening is designed to be long in the horizontal direction, as is preferred in this invention, the stresses are more evenly distributed and this minimizes chances for tears in the strapping device.

In yet another embodiment of the present invention, pipings are provided around each of the openings. The openings in the strapping device are typically made by cutting the fabric material used in making the strapping device. Fabric materials are typically made of fibers and the cuts will cause the fiber ends to appear around the edges of the opening. These loose fiber ends can cause potential problems, as fibers can then break and create loose fiber pieces in the process. Loose fibers can end up between the funnel shaped attachment and the breast, and this can cause loss of vacuum when the breast pump is in operation. The loose fibers can also end up between the funnel shaped attachment and the connecting devices of the breast pump which carry the milk into the containers. Even more dangerous is when the fiber pieces can end up in the milk itself, which can potentially choke or harm the baby if ingested. The use of piping around the edges of the opening prevents loose ends from getting free from the strapping device.

The use of piping around the openings provides further benefits as well. The ends of the openings are prone to tear easily, and the piping provides additional mechanical support to the strapping device material to reduce this tendency. Even more importantly, the pipings are highly efficient in distributing pressure, which is created both from the strapping action and from the vacuum action of the breast pump, to all regions around the nipples in the breasts. Pipings by design are thicker than the strapping device itself. This difference in thickness causes the pipings to be the main point of contact between the strapping device and the funnel shaped attaching device of the breast pump. The thickness of the piping can be increased or decreased by design to optimally achieve this pressure distribution to all regions around the nipples, where the milk ducts are located and which needs to be pressed in order to squeeze the milk optimally from the breasts.

In yet another embodiment of the present invention, the strapping device described here can also double up as a device to attach ice bags or cold materials to the breast. When the breast is engorged, the person experiences severe pains and the most common approach is to apply ice bags, frozen vegetable packets, or other cold materials to the breast to alleviate the pain. The strapping device described in this invention can be used for this purpose very easily and efficiently.

The foregoing description discloses exemplary embodiments of the present invention. It will be understood by those skilled in the art that the invention is not limited to the embodiments enclosed and numerous changes, modifications, substitutions, variations, and alternates are possible without departing from the scope and spirit of the invention, as defined by the following claims:

1. An improved strapping device for use with a breast pump, wherein the strapping device comprises a single piece long unit, a longitudinal axis running along a length of the single piece long unit, the single piece long unit having two ends, each end of said single piece unit having an attachment, wherein a maximum length of each attachment is substantially parallel to said longitudinal axis, wherein a width of each attachment is substantially perpendicular to said longitudinal axis, wherein said maximum length of each attachment is longer than said width of said attachment, wherein the attachments are capable of being connected together from an extended unconnected position to form a circular piece that can go around the users torso; wherein the strapping device has two openings, each of which is located, sized and shaped to support a funnel shaped attachment of the breast pump onto each breast, wherein a length of each of said two openings is substantially parallel to said longitudinal axis, wherein a width of each of said two openings is substantially perpendicular to said longitudinal axis, wherein said length of each of said two openings is substantially longer than said width of each of two said openings, wherein each of said two openings is formed in an elongated shape, said elongated shape formed by two long sides substantially parallel to said longitudinal axis arid two shorter end sides, substantially perpendicular to said longitudinal axis, wherein a gap in said width direction exists between said two long sides exists, such that there is no contact between said two long sides when said single long piece unit is in said extended unconnected position, wherein said two shorter end sides are formed substantially in the shape of a semi-circle, wherein each of the openings has piping all around them, wherein the single piece unit also has piping all around a periphery of the single long piece unit and wherein the piping all around the periphery of the single piece long unit and the openings is thicker than a thickness of a material of said single piece long unit.

2. The strapping device of claim 1, wherein the two attachments at their ends comprise mating parts of hooks, clips, buttons, press buttons, zip, hook and loop fastener, or similar attaching mechanisms.

3. The strapping device of claim 1, wherein the two openings are spaced 5 to 50 cm apart; as measured from wherein a vertical center of each opening intersects, a horizontal center of each other opening.

4. The strapping device of claim 1, wherein the two openings are spaced 10 to 40 cm apart, as measured from wherein a vertical center of each opening intersects a horizontal center of each other opening.

5. The strapping device of claim 1, wherein each of the two openings are between 2 and 20 centimeters in length.

6. The strapping device of claim 1, wherein each of the two openings are between 5 and 15 centimeters in length.

7. (canceled)

8. The strapping device of claim 1, wherein the piping around the two openings each has a color, the color of the piping around each opening being different than that of a color the of said single piece long unit.

9. An improved strapping device for use with a breast pump, wherein the strapping device comprises a single piece long unit, a longitudinal axis running along a length of the single piece long unit, wherein the single piece long unit has two openings, each of which is located, sized and shaped to support a funnel shaped attachment of the breast pump onto each of the breasts, wherein a length of each of said two openings is substantially parallel to said longitudinal axis, wherein a width of each of said two openings is substantially perpendicular to said longitudinal axis, wherein said length of each of said two openings is substantially longer than said width of each of two said openings, wherein each of said two openings is formed in an elongated shape, wherein ends of each elongated shape are formed substantially in the shape of a semi-circle, wherein each of the said openings having piping all around them and wherein the single piece unit has piping all around a periphery of the single piece long unit.

10. The strapping device of claim 9, wherein the two openings are spaced 5 to 50 cm apart, as measured from wherein a vertical center of each opening intersects a horizontal center of each other opening.

11. The strapping device of claim 9, wherein the two openings are spaced 10 to 40 cm apart, as measured from wherein a vertical center of each opening intersects a horizontal center of each other opening.

12. The strapping device of claim 9, wherein each of the two openings are between 2 and 20centimeters in length.

13. The strapping device of claim 9, wherein each of the two openings are between 5 and 15 centimeters in length.

14. (canceled)

15. The strapping device of claim 9, wherein the piping around the two openings each has a color, the color of the piping around each opening being different than that of a color the of said single piece long unit.

16. An improved strapping device for use with a breast jump, wherein the strapping device comprises a single piece long unit, a longitudinal axis running along a length of the single piece long unit, the single piece long unit having two ends, each end having an attachment, wherein a maximum length of each said attachment is substantially parallel to said longitudinal axis, wherein a width of each attachment is substantially perpendicular to said longitudinal axis, wherein said maximum length of each attachment is longer than, said width of said attachment, said elongated shape formed by two long sides substantially parallel to said longitudinal axis and two shorter end sides substantially perpendicular to said longitudinal axis, wherein a gap in said width direction exists between said two long sides exists, such that there is no contact between said two long sides when said single long piece unit is in said extended unconnected position, wherein the attachments are capable of being connected together to form a circular piece that can go around the users torso; wherein the strapping device has two openings, each of which is located, sized and shaped to support a funnel shaped attachment of the breast pump onto each of the breasts, wherein a length of each of said two openings is substantially parallel to said longitudinal axis, wherein a width of each of said two openings is substantially perpendicular to said longitudinal axis, wherein said length of each of said two openings is substantially longer than said width of each of two said openings, wherein each of said two openings is formed in an elongated shape, wherein ends of each elongated shape are formed substantially in the shape of a semi-circle and wherein the strapping device can also be used to attach cold materials to the person's breast specifically to help alleviate breast engorgement, wherein each of the said openings having piping all around them, wherein the single piece unit has piping all around a periphery of the single piece long unit, wherein the piping all around the periphery of the single piece long unit and the piping around the openings is thicker than a thickness of a material of said single piece long unit.

17. The strapping device of claim 1, wherein a material of the single piece long unit is composed of a mix of nylon, lycra®, cotton and neoprene.

18. The strapping device of claim 9, wherein a material of the single piece long unit is composed of a mix of nylon, lycra®, cotton and neoprene.

19. The strapping device of claim 16, wherein a material Of the single piece long unit is composed of a mix of nylon, lycra®, cotton and neoprene.

20. The strapping device of claim 1, wherein a material of the single piece long unit is composed of spandex®.
